(19) United States
(12) Patent Application Publication
(10) Pub. No.: US 2008/0312511 A1
(43) Pub. Date: Dec. 18, 2008

(54) ANIMAL HEALTH MONITORING SYSTEM AND METHOD

(75) Inventors: Lloyd Osler, Edmonton (CA); Mark Vernon Fedorak, Edmonton (CA); Tadeusz Kazmierczak, Edmonton (CA); Duncan Campbell, Edmonton (CA); John-Michael Bernard Carolan, Edmonton (CA); Rodney Ridley, Edmonton (CA); Joseph Wheeler, Edmonton (CA); Reginald Schmidt, Edmonton (CA); Corinne Schmidt, Edmonton (CA); Garry Cardinal, Edmonton (CA); Kevin Cyca, Edmonton (CA); Geoffrey Chambers, Edmonton (CA); Jeffrey Min Yao Huang, Edmonton (CA); Edmond Hok Ming Lou, Edmonton (CA); Bruce Brososky, Edmonton (CA); Christopher Charles Kirchen, Edmonton (CA); Donald Mullen, Edmonton (CA)

Correspondence Address: EDWARD YOO C/O BENNETT JONES, 1000 ATCO CENTRE, 10035 - 105 STREET, EDMONTON, ALBERTA, AB, T5J3T2, CA

(73) Assignee: ALBERTA RESEARCH COUNCIL INC., Edmonton (CA)

(21) Appl. No.: 11/763,382

(22) Filed: Jun. 14, 2007

(51) Int. Cl. A61B 5/01; A61B 5/00

(52) U.S. Cl. 600/300; 600/555

(57) ABSTRACT

The invention provides a real-time method and computer-implemented system of monitoring animal health comprising sensing at least one physical characteristic by means of an active physical sensor attached to the animal, and sensing at least one activity of the animal by means of an activity sensor attached to the animal; positioning an active activity signal generator in the environment, such that the activity signal generator is associated with an activity, gathering physical characteristic data and activity data, and wirelessly transmitting all such data to a network receiver/converter, in real-time; converting all such data if necessary, and transmitting all such data over a computer network to one or more users, in real-time.

### FIELD OF THE INVENTION

The present invention relates to a real-time method and computer-implemented system for the monitoring of animal health.

### BACKGROUND

The conventional method of monitoring animal health consists of performing visual inspections until clinical symptoms of illness are displayed, and then treating those symptoms. However, visual inspections may be too infrequent and a human observer may miss obscured symptoms of disease or illness. As well, by the time an animal is visibly sick, it is often too late to reverse the onset of illness or prevent an outbreak of disease to other animals, and an animal's weight gain may have already been compromised.

It is known to use sensors to monitor animal health; for example, United States Patent Publication No. 2002/0010390 to Guice et al. describes a system in which wireless “smart tele-sensor” elements are implanted in the animal and programmed to transmit an alert signal. However, implants are invasive and may be uncomfortable for the animal. Further, additional surgery may be inconveniently required to remove components for maintenance, repair or to prevent device components from entering the food chain.

It is known to use electronic RFID tags which are externally attachable to an animal, hence unobtrusive and readily mounted and removed. RFID tags utilize an embedded passive tag having a unique identifier. The tag identifies the origin of a particular animal in the event of a disease outbreak in order to trace the animal back to a specific herd; however, the current RFID systems require hand-held or gated systems which are expensive, error-prone, marginally effective, not scalable and provide no real benefit to producers.

It is known to use GPS-based monitoring systems for monitoring animals; for example, U.S. Pat. Nos. 6,375,612 and 6,569,092 to Guichon et al. describe a system for tracking the movement of animals from location to location during processing. Animals are fitted with a collar or ear tag to which a data collection and transmission unit including a GPS receiver is attached. An interrogator is programmed to read the data collection and transmission unit, and conveys data to a processor. This system, however, is of limited use to producers since only GPS positional data is collected.

It is known to monitor feeding or watering activities in order to assess an animal's health; for example, U.S. Pat. No. 6,427,627 to Huisma describes a system including antennas located at selected spaced intervals along an elongate feed or drinking trough to detect feeding and watering. The animal has an implanted or attached passive transponder having an identification code. An electronic control system transmits an electronic signal sequentially to each antenna so that each activated antenna emits a signal. Any passive transponder sufficiently adjacent to the activated antenna receives the signal and generates a return electronic signal which is sent to the activated antenna. This system, however, only indirectly and after the fact assesses an animal's health through feeding or watering activities.

Therefore, there is a need in the art for methods and systems of remotely monitoring and recording, in real-time, data that is indicative of an animal's health, in such a way that an intervention may be initiated on a timely basis.

### SUMMARY OF THE INVENTION

The present invention provides a real-time method and computer-implemented system of monitoring animal health. Therefore, in one aspect, the invention comprises a system for monitoring at least one physical characteristic and at least one activity of an animal within an environment, said system comprising:
(a) at least one active physical sensor capable of detecting at least one physical characteristic or at least one environmental characteristic, or both a physical characteristic and an environmental characteristic,
(b) at least one active activity sensor for detecting an activity signal generator associated with an activity;
(c) a transceiver operatively connected to the at least one sensor and the at least one transceiver module sensor, for receiving data from at least one sensor and at least one transceiver module sensor, and transmitting the data using a wireless protocol, in real-time;
d) a receiver/converter for receiving data received from the transceiver, and for transmitting the data over a computer network, in real-time.
   In another aspect, the invention may comprise a method for monitoring at least one physical characteristic and at least one activity of an animal within an environment, said method comprising the steps of:
a) sensing at least one physical characteristic by means of an active physical sensor attached to the animal, and sensing at least one activity of the animal by means of an activity sensor attached to the animal;
b) positioning an active activity signal generator in the environment, such that the activity signal generator is associated with an activity,
c) gathering physical characteristic data and activity data, and wirelessly transmitting all such data to a network receiver/converter, in real-time;
d) converting all such data if necessary, and transmitting all such data over a computer network to one or more users, in real-time.

### BRIEF DESCRIPTION OF THE DRAWINGS

In the drawings, like elements are assigned like reference numerals.

FIG. 1 is a schematic representation of one embodiment of the present invention.

FIG. 2 is a block diagram of one embodiment of a physical sensor.

FIG. 3 is a block diagram of one embodiment of a transceiver module.

FIG. 4 is a block diagram of one embodiment of an activity signal generator.

FIG. 5A is a flowchart showing one embodiment of a method for detecting and transmitting signals for an associated activity. FIG. 5B is a flowchart showing one embodiment of further processing of the watering activity interrupt flag.

FIG. 6 is a flowchart showing one embodiment of a method for processing and analyzing physical attribute and activity data.

### DETAILED DESCRIPTION OF PREFERRED EMBODIMENTS

The present invention relates to a real-time method and system for monitoring and recording data indicative of animal health, remotely and in real-time. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

Those skilled in the art will realize that components of one embodiment of the invention described herein may be realized in suitable hardware, firmware, software, firmware/software, and/or firmware/software logic blocks, objects, modules or components or in combination thereof.

As used herein, “real-time” or a “real-time method” refers to the reporting of data representative of live events simultaneously with their occurrence or so near simultaneous that the delay does not significantly impact the operation of subsequent events. Preferably, a real-time method operates continuously, or in continuous periods. Since data representative of the live events are gathered and made available as the events occur, the devices mediating data collection are continuously active during the same time frame.

As used herein, “operatively connected” means, in the case of hardware,—an electrical connection, e.g. wire or PCB trace, for conveying electrical signals, or in the case of firmware or software, a communication link between the processor (which executes the firmware—i.e. operating under stored program control—or software) and another device for transmitting/receiving messages or commands.

As used herein, an “active device” means a device that measures one or more physical properties and that also contains an energy source. The energy source allows the active device to obtain its readings without rigid time constraint and to transmit these readings to a receiving device at a distance. An active device is operative by means of circuitry which actively mediates or engages in one or more functions. An active device is distinguishable from a passive device, which is defined as a device that does not perform any active function, except when energized by an external energy source, whether by physical connection or by RF field. A passive device is therefore constrained to transmitting its readings when in proximity to such an energy source. For example, a RFID device is a passive device which sends stored data only when energized by an external field, and which does not include any capability of actively sensing and storing data.

As used herein, “geotemporal” means event-based (coincident geographical and time stamped) data elements as related to a database or repository. Such a geotemporal data structure provides for analysis of data and information both back and forward in time, within the context of any one or multiple geographical locations and to map such information. This geotemporal data structure enables the full traceability and tractability of a given animal, including all its production and health data, from birth to slaughter.

As used herein, “geotemporal historical analysis” means the ability to analyze data backwards in time relevant to a geo-position, or analyze data relative to a geo-position or positions for a specific time. An example would be the ability to go back in time, and connect geographically, two or more animals that had been in contact or connected in some way, at exactly that time, or determine if two animals had ever been in contact, at any time.

As is used herein, “geotemporal predictive analysis” means the ability to analyze data forward in time, relative to a geo-position or positions; or analyze data relative to a geo-position or positions for a specific time. An example would be the ability to project the next likely outbreak of the disease relevant to future time and location, and further based on lapse time analysis, predict the scope or scale of the outbreak.

In general terms, as shown in FIG. 1, a system (1) of the present invention includes a physical sensor (10) which is worn by an animal to be monitored for detecting at least one physical characteristic of the animal. The physical sensor (10) is operatively connected with a transceiver module (12), also worn by the animal. The transceiver module (12) also communicates with an activity signal generator (14) which is positioned in the environment and associated with an activity, and which communicates with the transceiver module (12). The transceiver module (12) comprises a wireless transceiver (38), which transmits data to a network receiver/converter (16). The receiver/converter (16) receives the physical attribute data from the wireless transceiver (38) using a wireless technology which includes a physical transmitter and receiver as well as a wireless protocol. Following processing or conversion of the data if necessary, the network receiver/converter (16) transmits the data over a computer network (18) to one or more users (20). Components of one embodiment of the system are described in greater detail as set out below.

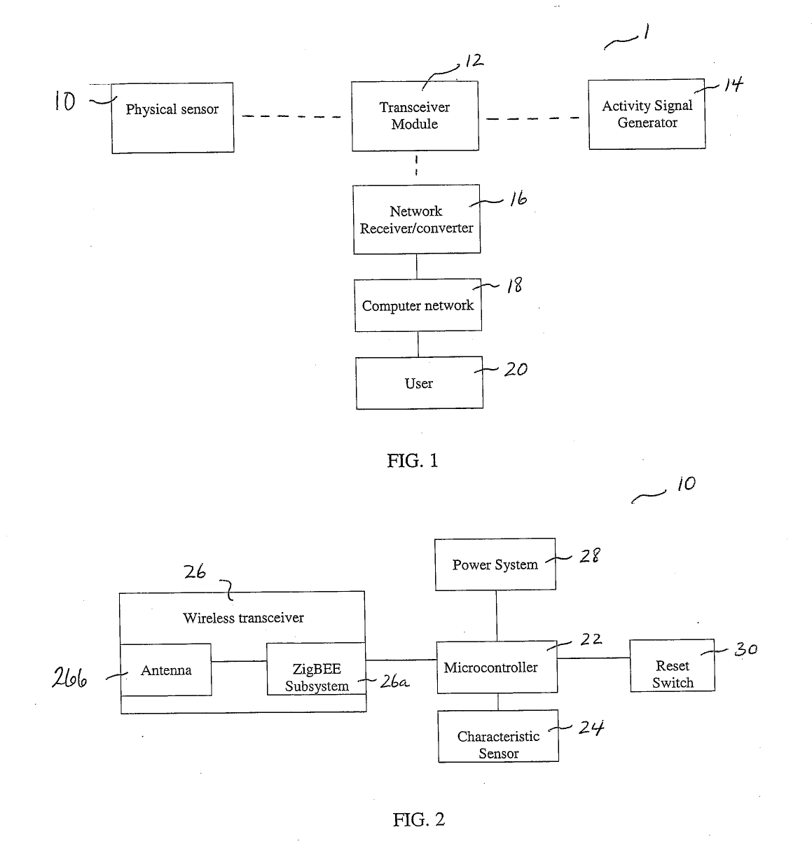

As illustrated in FIG. 2, one embodiment of the physical sensor (10) includes a microcontroller (22) which is operatively connected to a characteristic sensor (24) and a wireless transceiver (26). In one preferred embodiment, the microcontroller (22) has a serial peripheral interface and analog-to-digital capabilities in order for it to communicate with the characteristic sensor (24) and the wireless transceiver (26); sufficient code space to incorporate the firmware required for operation; the ability to enter a very low power mode to minimize consumption; and real-time clock capabilities. As an example, an AVR microcontroller manufactured by Atmel is suitable.

The characteristic sensor (24) is capable of measuring a physiological parameter, for example, body temperature, blood oxygen levels or heart rate; or an environmental parameter, for example, ambient temperature, atmospheric pressure, relative humidity, wind speed or system status; or both a physiological and an environmental parameter. The physical sensor (10) may comprise a plurality of characteristic sensors (24) for different physiological and environmental parameters.

The wireless transceiver (26) can comprise any wireless transmitter such as, for example, a non-protocol based RF transmitter, a Bluetooth transmitter, a wireless Universal Serial Bus (USB) transmitter, or a ZigBEE® transmitter. In one preferred embodiment, a ZigBEE® transmitter (26a) such as a Chipcon chipset and antenna (26b) are used. A small chip antenna with a peak gain of 0.5 dBi manufactured by Lynx Antenna Systems is suitable. The microcontroller (22) is powered by a power system (28), for example, a battery. A non-rechargeable, lithium coin cell battery such as a CR2032 battery (Panasonic) supported by standard Keystone coin cell battery holder are suitable.

Reset means (30) is included to restart the hardware and to resynchronize the physical sensor (10) with the wireless transceiver (38). Resynchronization is achieved by ensuring that the transmission window is long enough to maintain synchronicity between the physical sensor (10) and the wireless transceiver (38). If either device misses a transmission window, both devices turn on for a longer period of time, such as twice the length of time, to increase the probability of synchronization. If synchronization is still not achieved, the physical sensor may attempt to connect directly to the network receiver/converter (16). For example, if the physical sensor (10) is unable to communicate with the wireless transceiver (38) after ten attempts, then the physical sensor (10) enters a low power mode in which it tries to establish a connection intermittently and directly to the server to resynchronize with the wireless transceiver (38).

The physical sensor (10) is incorporated with additional components (not shown) well known in the art, including, for example, standard circuit components such as buffers, capacitors, voltage regulators, inductors and resistors.

In one embodiment, the physical sensor (10) is incorporated in the form of an ear tag to be worn by the animal. The described components of the physical sensor (10) are housed within a plastic casing (not shown) composed of two halves sealed by a rubber gasket and connected together with suitable attachment means such as aluminum pins or screws. In one embodiment, the casing has an opening or a deformable area on one side to enable depression of an internal pin which resets the internal electronics. Additional pins are included to pierce through the animal's ear and enter a polyethylene, or bio-safe rubber plug on the back of the ear to secure the ear tag.

In one embodiment, both the physical sensor (10) and the transceiver module (12) are contained in the same package. This combined package could be an ear tag, a collar module, a brisket tag, implantable sensor or affixed to the animal in some other way. In this case, the connection between the physical sensor and the transceiver module may not be wireless, but may comprise a physical connection.

In one embodiment, the physical sensor (10) or the transceiver module (12), or both, may contain a means which can be used to identify the animal should a symptom of illness be detected by the system. This mechanism for example could be a visual indicator such as an LED light, or an auditory indicator such as a piezoelectric alarm. The mechanism may activate continuously, or in a pulsed state. Alternatively, the mechanism may comprise a RF signal generator that could be used in conjunction with a handheld device to determine the location of the animal using the properties of direction finding and possibly triangulation.

In one embodiment, the piercing pins are made of a metal or other material having excellent heat conduction properties, such as aluminum. As a result, the aluminum pin may be used to sense the animal's body temperature.

In one embodiment, the physical sensor (10) is incorporated in the form of an implant which is positioned subcutaneously or within the body of the animal. The implant is preferably biocompatible and non-toxic, or enclosed in a biocompatible case, generating no significant undesirable host response.

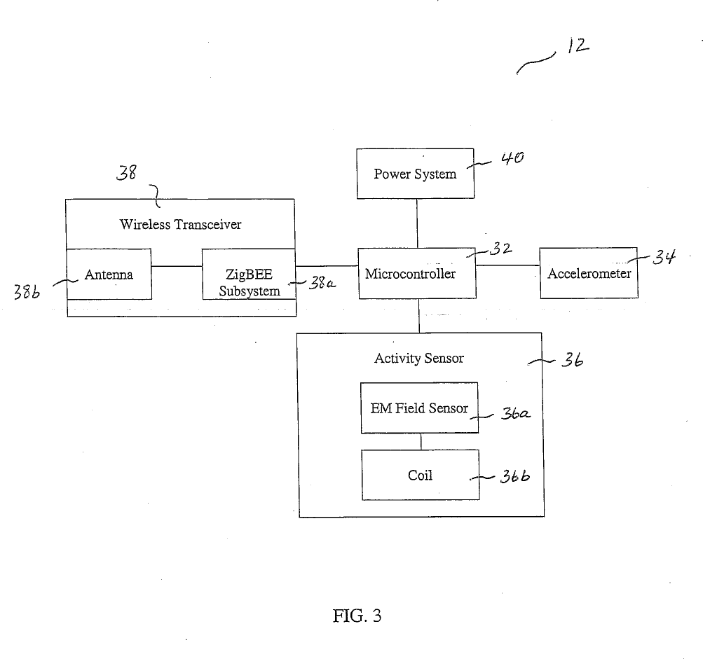

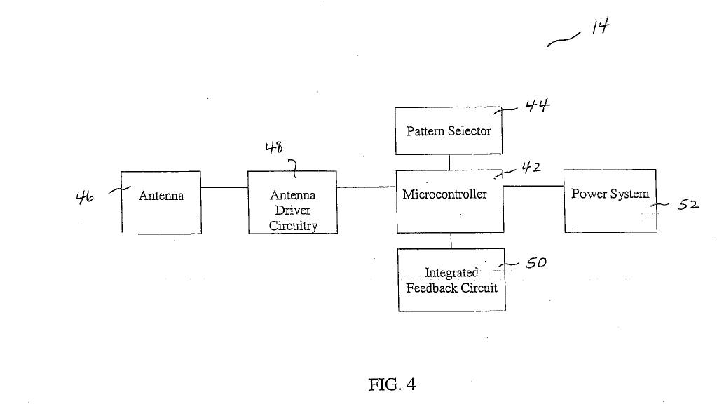

As illustrated in FIG. 3, one embodiment of the transceiver module (12) includes a microcontroller (32) which is operatively connected to an accelerometer (34), an activity sensor (36) and a wireless transceiver (38). In one preferred embodiment, the microcontroller (32) has serial peripheral interface capabilities to enable communication with the wireless transceiver (38); three analog to digital converter channels in order to sample three axes of motion data; general purpose I/O pins for communication with the activity sensor (36); sufficient code space to incorporate the firmware required for operation; the ability to enter a very low power mode to minimize consumption; and real-time clock capabilities. As an example, an AVR microcontroller manufactured by Atmel is suitable.

The accelerometer (34) detects movement of the animal in at least two, and preferably three axes of motion. Any standard accelerometer which can measure a suitable range of acceleration in either two or three dimensions can be used. In an exemplary embodiment, an accelerometer (34) senses up to 2 g in the X and Y directions and up to 1 g in the Z direction.

The activity sensor (36) detects and distinguishes the duration and frequency of one or more activities, for example, at least feeding and watering. In one embodiment, the activity sensor (36) comprises an electromagnetic (EM) field sensor (36a) and a coil (36b) to detect feeding and watering activities. The activity signal generator (14) generates electromagnetic fields representative of feeding or watering which are picked up by the coil (36b) and communicated to the EM field sensor (36a). The EM field sensor (36a) distinguishes between the two different fields and reports them separately.

The microcontroller (32) also communicates with the wireless transceiver (38) which includes a wireless transmitter. The wireless transmitter can comprise any wireless transmitter such as, for example, a non-protocol based RF transmitter, a Bluetooth transmitter, a wireless USB transmitter, or a ZigBEE® transmitter. In one preferred embodiment, a ZigBEE® transmitter (38a) such as a Chipcon chipset and antenna (38b) are used. A Titanus antenna with a peak gain of 4.4 dBi manufactured by GigaANT is suitable. In an exemplary embodiment, the antenna (38b) is capable of sending data up to the physical dimensions of the animal's environment, which may be a feed pen. Typically a range of about 100 m will be sufficient for animals inside of a feedlot. The wireless transceiver (38) receives physical attribute data from the physical sensor (10) and transmits the data to the network receiver/converter (16). The microcontroller (32) is powered by a power system (40), for example, a battery. A non-rechargeable, 3 V lithium battery such as CR2 (Energizer or Lisun) and a standard Keystone battery holder are suitable.

The transceiver module (12) is incorporated with additional components well known to those skilled in the art (not shown) including, for example, standard circuit components such as buffers, capacitors, voltage regulators, inductors and resistors.

In one embodiment, the transceiver module (12) is incorporated in the form of collar to be worn by the animal. In one embodiment, the described components of the transceiver module (12) are housed within a plastic casing (not shown) composed of two halves sealed with a rubber gasket and connected together with suitable attachment means such as stainless steel double hex plastic screws. A nylon strap and two D-ring type strap connectors sewn into one end facilitate attachment around the neck of the animal.

FIG. 4 illustrates one embodiment of the activity signal generator (14) which includes a microcontroller (42), a pattern selector (44), an antenna (46), associated antenna driver circuitry (48), an integrated feedback circuit (50) and a power system (52). Each activity signal generator (14) generates a distinctive signal for an associated activity, for example, feeding, watering, salt intake, milking station visits, and shade locations. The signal generator (14) may be identical for each activity, except for one differentiating aspect which permits the activity sensor (36) to differentiate between the different activities.

The microcontroller (42) can be, for example, an AVR microcontroller manufactured by Atmel. The microcontroller (42) communicates with the pattern selector (44) to generate a low frequency RF pattern which is emitted by the antenna (46) and detected by the activity sensor (36) for interpretation as a particular activity (for example, either the feeding pattern or watering pattern).

In one embodiment, the antenna (46) is formed of an insulated or non-insulated copper wire with a single or multiple of winds depending on the individual characteristics of the location to be monitored. The winds may be sealed inside of a plastic conduit. The antenna (46) emits a radio frequency modulated field to a variable distance depending upon the power level at which the antenna driver circuitry (48) is set. Preferably, the power level of the antenna (46) is kept constant through a continuous measurement through an integrated feedback circuit (50). The transmitted field can consist of one of many different patterns which can be toggled by the pattern selector (44). In one embodiment, the activity signal generator (14) is powered from 120VAC which is brought down to 12VDC through a step down transformer; however, any power source capable of supplying the current required would be acceptable.

The microcontroller (42) is used in conjunction with the EM field sensor (36b) of the transceiver module (12) to create a low frequency wakeup signal and control the emitted signal strength as required. This signal may be detected for example, by the activity sensor (36) with time stamps in order to track feeding or watering patterns. The microcontroller (42) creates and modulates the carrier signal, which is transmitted from an antenna (46) with a signal strength, which is also controlled by the microcontroller (42) via a digital potentiometer or D/A converter part of the antenna driver circuitry (48).

In one embodiment, the activity signal generator (14) is connected to two separate receiver antennae as part of a feedback mechanism (50), one of which is mounted within the emitter antenna (46) coil in a region of high signal power, and one that is mounted outside the emitter antenna (46) coil in a region of low signal power. The feedback receiver (50) monitors both antennae non-simultaneously, continuously alternating the antenna being monitored. The purpose of the integrated feedback circuit (50) is to adjust the emitted signal strength so as to maximize the signal reception on the near antenna and minimize reception on the far antenna. In the field, this design is used to ensure that the emitted signal is sufficient, such that the activity signal generator (14) detects the signal if the animal feeds, but does not detect any false alarms.

Upon power-up, the activity signal generator (14) is configured to scan the feedback circuit (50) antenna repeatedly until it detects a stable carrier signal within a given frequency range. Once the carrier signal has been detected, the microcontroller (42) monitors the amplitude envelope of the carrier signal and waits for a pre-defined wakeup sequence to occur. Under normal operation, once the wakeup sequence has been detected, the microcontroller (42) goes into receive mode and waits for further transmissions. In one embodiment, the microcontroller (42) is reset by a signal from the feedback circuit (50) upon completion of the wakeup sequence so that the feedback circuit (50) again searches for a repeat of the wakeup sequence. In this way, by using the microcontroller (42) to transmit an endlessly repeating series of wakeup sequences and monitoring whether the wakeup sequence has been picked up by the feedback circuit (50), the program is able to measure quantitatively the antenna reception, and modify the signal strength accordingly.

In one embodiment, the wakeup sequence consists of a start bit (0), an 8-bit wakeup code, and a stop bit (1). Two different 8-bit patterns are defined which could later be used to differentiate between different emitter antennae (46), thus differentiating between different activities. The carrier signal is a square-pulse train of a specified frequency, and the bit time for the modulating signal is 256 μs. Manchester coding is used to define the modulating signal bits. Thus, a “0” is denoted by a falling edge, the envelope of the carrier signal is high for half the bit time and low for the second 128 us. Similarly, a “1” is denoted by a low envelope for the first half of the bit time and a high envelope for the second half. In addition to the actual wakeup sequence, there is a preamble during which the envelope is high. As well, the activity signal generator (14) may require initialization time after it is reset; thus, there is a lull after the wakeup sequence during which time the envelope level is low (i.e., the carrier signal is completely suppressed). For this reason, rather than storing the actual wakeup sequence in the microcontroller memory, it is easier to store a table denoting the level of the envelope (Bit Value) and the length of time that this level is sustained for (Delay Value, in μs).

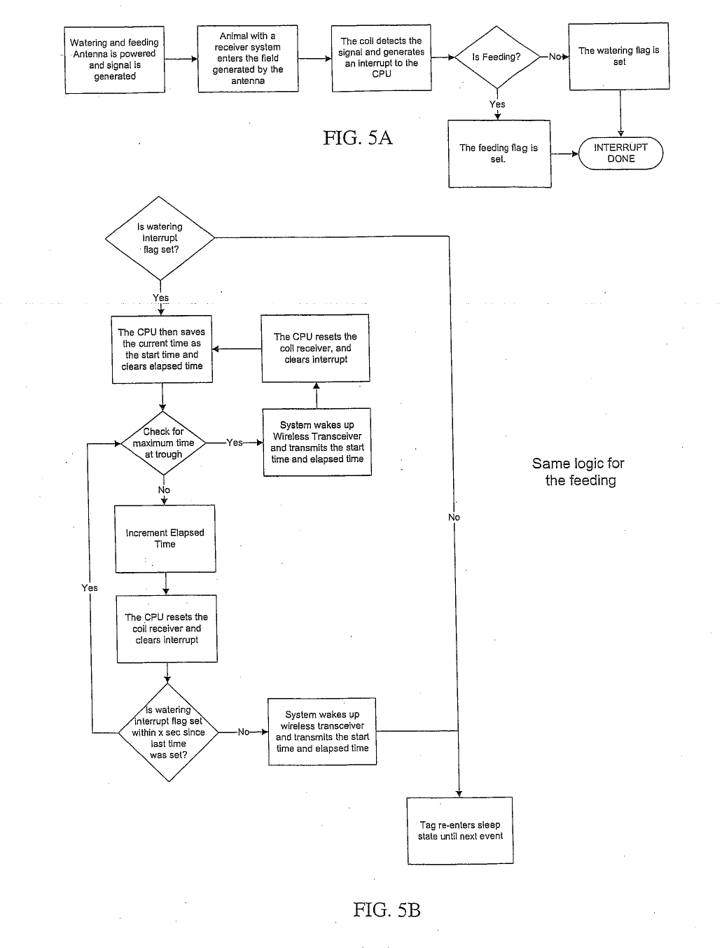

FIG. 5A illustrates one embodiment of the method for detecting and transmitting signals for an associated activity. When the watering and feeding antennas are powered and the respective signals are generated, and an animal wearing a transceiver module (12) comprising an activity sensor (36) enters the field, the activity sensor detects the signal and generates an interrupt to the transceiver module controller (32). If the signal is recognized as being associated with feeding, then a feeding flag is set. If the signal is recognized as being associated with watering, then a watering flag is set. This can be expanded for other patterns that might indicate the other activities in which the activity sensor system is used.

FIG. 5B illustrates one embodiment of further processing of the watering activity interrupt flag. The processing of a feeding activity interrupt flag or other activity flag may be identical. As shown, if the watering interrupt flag is set, in other words, the animal has entered the field generated by the watering activity signal generator (14) and the signal has been interpreted as being associated with watering, then the transceiver module controller (32) will save the current time as a start time, and clears the elapsed time. If the watering interrupt flag is not set, then the activity sensor (36) reenters a sleep or standby mode. The elapsed time is then incremented so long as a maximum elapsed time has not been exceeded. The watering activity interrupt is then cleared. If the interrupt flag is set again within a predetermined length of time since the last time it was set, in other words, the animal is still within the field of the watering activity signal, then the elapsed time is incremented. If the interrupt flag is not reset again within the predetermined length of time, then the controller (32) wakes up the wireless transceiver (38) and transmits the start time and elapsed time to the network receiver/converter (16). If a maximum elapsed time is reached, then the start time and elapsed time is also transmitted, the interrupt is cleared. It is also possible to have the start and elapsed times of the activity event sent at the next scheduled data transmission.

In one embodiment, the described components of the activity signal generator (14) are housed within a water tight box (not shown) which is also connected through a sealed connection to the antenna which is housed inside of PVC conduit. The activity signal generator (14) is positioned inside of or adjacent to, for example, a feeding trough or water bowl. The emitter antennae (46) are affixed with metal pipe supports around the feeding trough and watering bowl to wooden planks. The wooden planks are attached to metal bars which surround the feed bunks and watering bowl.

The network receiver/converter (16) functions to receive data transmitted by the wireless transceiver (38) and, if necessary, convert to use in a computer network implemented by end users. In one embodiment, the network receiver/converter (16) comprises a ZigBEE-to-Ethernet converter. For example, a Rabbit 2000 TCP/IP module which includes a ZigBEE® subsystem (Chipcon) and antenna (Titanus antenna with a peak gain of 4.4 dBi manufactured by GigaANT) is suitable. The network receiver/converter (16) thus comprises code or firmware consisting of, for example, a ZigBEE® packetization code module, an Ethernet or TCP/IP packetization code module, associated connection persistence modules, and the various required conversion functions. In one embodiment, power for the network receiver/converter (16) is provided from power over Ethernet. A standard industrial temperature range system consisting of two modules (i.e., one to combine the power and data signal and the other to split the power and data signals) is suitable.

In operation, the network receiver/converter (16) accepts and establishes a ZigBEE® connection to the wireless transceiver (38). The network receiver/converter (16) receives all the physical attribute and activity data gathered by the transceiver module (12), strips off the ZigBEE® packet protocol packaging, and repackages the data as IP packets. The IP packets are subsequently sent over an Ethernet connection using a Cat5e cable and through a TCP connection to the server. The receiver/converter (16) then takes data from the TCP connection with the server, removes the IP packaging from the payload data and then packages the data as a ZigBEE® packet and sends it to the ZigBEE® chip for transmission to the transceiver module (12). The transmissions to the transceiver module (12) may consist of but are not limited to firmware upgrades, parameter updates included changes to the sample rates, and sample intervals, reset requests, calibration values, receipt of data acknowledgements, and other configuration data.

In one embodiment, the described components of the receiver/converter (16) are housed within a suitable waterproof, impact resistant plastic casing (not shown), for example a standard enclosure from Pactec or Hammond Manufacturing. The enclosure is modified by drilling holes in the back of the box to facilitate mounting of the internal components and of the enclosure itself. An additional hole is drilled through the base for passage of the Ethernet cable. Should a wireless Ethernet connection be used, then no additional holes will be required. An additional hole may be required for a power connection. Washers are placed on the interior of the box to seal the holes. Suitable mounting means can include, for example, rustproof bolts which are sealed with washers. In the feedlot, the box is mounted on a board at sufficient height to be out of reach of animals and at a sufficient distance from metallic devices which might interfere with optimum signal transmission and reception.

The command and control software for the system (1) is designed to facilitate several functions, including, for example, real-time monitoring of all components; a display of real-time event based data recorded from the physical sensor (10) and transceiver module (12); input and update of production data (for example, addition of an animal to a farm); search/retrieval of an individual animal by an identifier, pen or other search parameters; identifying a treatment for each animal and recording the treatment; and an integrated real-time search by mapping animals across farm and other locations.

Analysis of the data may be made by using established diagnostic indicators, or known associations with environmental factors. For example, an elevated body temperature may be indicative of a fever, which may indicate an infectious disease. Increases or decreases in watering, feeding or other activity patterns may also be indicative of animal health.

In one embodiment, the software components may include prolog functions libraries for real time input/output; compileable C functions libraries for CGI interfaces, MySQL/PHP/C(CGI) interface; InetD/C/Prolog/SQL interface; Prolog/C/shell/Curl HTTP transport interface; and Prolog/C/Time/Spacemap; and ReventCarta™ (OVISTECH, real-time system including search and analysis functions and mapping functions). All data are collected as events into a geotemporal database. Consequently, the data collected are not limited to the confines of a farm alone, but to any geographical area to which the data may apply. In one embodiment, with regard to animal disease tracking and management, the system contains the capability and capacity to function across a farm, a region, or a wider geographical area. Further, the system also provides for access to and use of the data as the animal passes from one producer to another with full tracking and trace back capability. In one preferred embodiment, the system may provide access anytime anywhere and is implemented in HTML. It runs through any current web browser with a minimum of hardware requirements. Additionally, user access to the system may be provided through any device with Internet access and processing ability, such as a PDA, a smart phone or any other hand held device which supports Internet browsing capability.

The data may also be analyzed on a geotemporal historical basis. Data may be reviewed backwards in time, or a specific period in time, relevant to a geo-position. For example, a user may go back in time, and connect geographically, two or more animals that had been in contact or connected in some way, at exactly that time, or determine if two animals had ever been in contact, at any time. Furthermore, the data could then be used in geotemporal predictive analysis. For example, a user could project the next likely outbreak of the disease relevant to future time and location, and further based on lapsed time analysis, predict the scope or scale of the outbreak.

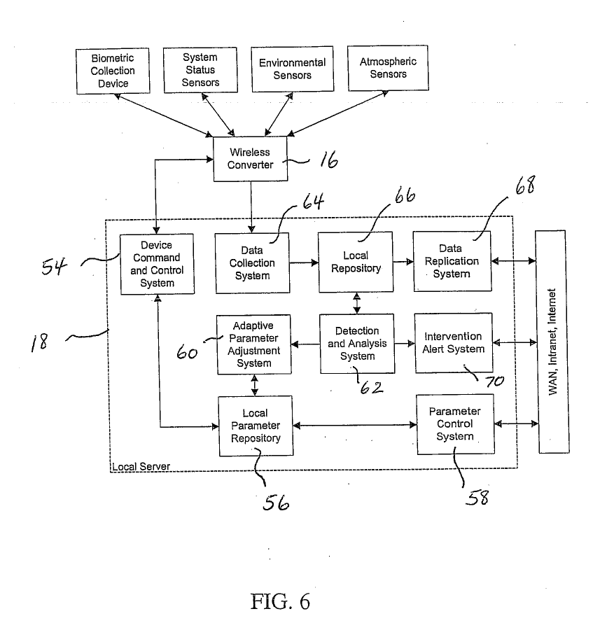

FIG. 6 illustrates a schematic block diagram of one embodiment of a system for processing and analyzing physical attribute and activity data. The network receiver/converter (16) receives the data and if necessary, coverts the data to use, first in a local server (18), and subsequently by end users over a computer network, such as a WAN, LAN or the Internet. In FIG. 6, a physical sensor (10) as described above may comprise a biometric collection device, an environmental sensor, an atmospheric sensor, or a system status sensor. The data received by the network receiver/converter may also include the activity sensor data described above. In one preferred embodiment, the local server (18) runs an operating system with the ability to interface with a network, for example, Ethernet, Wi-MAX, Wi-Fi or any other networking technology, and hosts the software which contains the systems as illustrated in FIG. 6.

In one embodiment, a device command and control system (54) is incorporated to command and control the devices of the system (1). The device command and control system (54) communicates with a local parameter repository (56) which comprises the local copy of all of the system (1) and device parameters. The local parameter repository (56) communicates with the parameter control system (58) which enables a user to view and modify various parameters for each individual deployed device. This system (58) can also override the recommendations from an adaptive parameter adjustment system (60).

The adaptive parameter adjustment system (60) comprises either a software application or a functionality of a larger software application which examines the results of a detection and analysis system (62) and may adjust, based on the system settings, the data collection parameters used by the devices. For example, this may include increasing the frequency of data collection for animals which appear to becoming ill or are already ill, and the reverse for healthy animals.

A data collection system (64) “listens for” or awaits data from the network receiver/converter (16), receives the data, and parses and formats the data for placement into a local repository (66). The local repository (66) comprises the local copy of all of the data collected by the system (1), as well as data inputted by the producers. All of the data is geographically and spatially-related since the date, time, and geographical location are stored with each data point. A data replication system (68) replicates the local repository (66) on a remotely located server or an end user system (not shown).

The detection and analysis system (62) comprises software which analyzes, in real-time, the data collected from the devices using detection algorithms to determine the current health of each animal in a herd. Based upon this determination, the detection and analysis system (62) signals the intervention alert system (70) which then alerts the producer that one or more animals require medical attention. Communication means including, for example, a terminal message, SMS text message, pager, electronic mail, telephone call, buzzer, siren, speaker, or any other technology are suitable for relaying an alert message.

Where power requirements for the system (1) or any component of the system is described, one skilled in the art will realize that any suitable power source may be used, including, without limitation, rechargeable and non-rechargeable batteries, photovoltaic cells and the like.

As will be apparent to those skilled in the art, various modifications, adaptations and variations of the foregoing specific disclosure can be made without departing from the scope of the invention claimed herein. The various features and elements of the described invention may be combined in a manner different from the combinations described or claimed herein, without departing from the scope of the invention.

1. A system for monitoring at least one physical characteristic and at least one activity of an animal within an environment, said system comprising:
(a) at least one active physical sensor capable of directly detecting at least one physical characteristic or at least one environmental characteristic, or both a physical characteristic and an environmental characteristic,
(b) at least one active activity sensor for detecting an activity signal generator associated with an activity;
(c) a transceiver operatively connected to the at least one physical activity sensor and the at least one activity sensor, for receiving data from either or both the at least one physical sensor and the at least one activity sensor and transmitting the data using a wireless protocol, in real-time;
d) a receiver/converter for receiving data received from the transceiver, and for transmitting the data over a computer network, in real-time.

2. The system of claim 1 wherein the at least one active physical sensor detects body temperature of the animal.

3. The system of claim 2 wherein the at least one active physical sensor also detects ambient temperature of the environment.

4. The system of claim 1 wherein the at least one active activity sensor comprises a first activity sensor which detects a first transmitter associated with feeding, and a second activity sensor which detects a second transmitter associated with watering.

5. The system of claim 4 wherein the first and second activity sensors comprise a single coil and circuit responsive to an electromagnetic field created by the activity signal generator.

6. The system of claim 1 wherein the at least one active physical sensor is contained within a first module, and the at least one activity sensor and the transceiver are contained within a second module.

7. The system of claim 1 wherein the at least one active physical sensor and the at least one activity sensor and the transceiver is contained within a unitary module.

8. The system of claim 6 wherein the first module is a tag comprising means to be attached to an appendage of the animal.

9. The system of claim 8 wherein the first module is an ear tag.

10. The system of claim 6 wherein the second module is attached to a neck collar.

11. The system of claim 1 wherein the transceiver is a non-protocol based RF transmitter, a wireless USB transmitter, or a wireless personal area network transmitter.

12. (canceled)

13. The system of claim 11 wherein the converter is a wireless personal area network to Ethernet converter.

14. The system of claim 1 further comprising an accelerometer for detecting movement of the animal, operatively connected to the transceiver.

15. A method for monitoring at least one physical characteristic and at least one activity of an animal within an environment, said method comprising the steps of:
a) sensing at least one physical characteristic by means of an active physical sensor attached to the animal, and sensing at least one activity of the animal by means of an activity sensor attached to the animal;
b) positioning an active activity signal generator in the environment, such that the activity signal generator is associated with an activity,
c) gathering physical characteristic data and activity data, and wirelessly transmitting all physical characteristic data and activity data to a network receiver/converter, in real-time;
d) converting all physical characteristic data and activity data if necessary, and transmitting all physical characteristic data and activity data over a computer network to one or more users, in real-time.

16. The method of claim 15 further comprising a step of sensing at least one environmental characteristic by means of an active physical sensor.

17. The method of claim 15 wherein the data is tagged by geographic origin and by time, and is stored in a geo-temporal database.
